(19) United States
(12) Patent Application Publication
(10) Pub. No.: US 2026/0088153 A1
(43) Pub. Date: Mar. 26, 2026

(54) ADAPTIVE COGNITIVE TRAINING AND COMBINED MUSIC LISTENING INTERVENTION

(71) Applicant: UNIVERSITY OF SOUTH FLORIDA, Tampa, FL (US)

(72) Inventor: Ashley CURTIS, Tampa, FL (US)

(21) Appl. No.: 19/334,577

(22) Filed: Sep. 19, 2025

Related U.S. Application Data
(60) Provisional application No. 63/697,242, filed on Sep. 20, 2024.

(51) Int. Cl. G16H 20/70; A63F 13/54

(52) U.S. Cl. CPC G16H 20/70; A63F 13/54; A63F 2300/638

(57) ABSTRACT

Systems and methods are provided herein for adaptive cognitive training and combined music listening intervention. In a method for treating cognitive dysfunction, the method includes: providing music to a user for a first time duration, and subsequently providing one or more cognitive training games to the user such that the user plays each of the one or more cognitive training games for up to a second time duration.

### CROSS-REFERENCE TO RELATED APPLICATION(S)

This application claims priority to U.S. Provisional Patent Application No. 63/697,242, filed on Sep. 20, 2024, the entire contents of which are incorporated herein by reference for all purposes.

### BACKGROUND

Mild cognitive impairment (MCI) affects about 16% of older adults in the United States. Approximately 45% of MCI patients experience insomnia, which only further exacerbates cognitive symptoms and disrupts mood. Both MCI and insomnia are associated with increased risk of Alzheimer's Disease (AD). Given that 10-15% of those with MCI develop AD each year, there is a need to treat cognition and associated disruptions during the critical window-before progression to AD.

Lack of pharmacological cognitive agents and the established negative impact of polypharmacy on cognitive and physiological functioning in aging adults has prompted research on behavioral interventions such as cognitive training, for example, a computerized program that trains mental activities.

Accordingly, a need exists for cognitive training to improve cognition and sleep. Also, a need exists for cognitive training to treat cognitive dysfunction, including cognitive impairment.

### SUMMARY

The following presents a simplified summary of the disclosed technology herein in order to provide a basic understanding of some aspects of the disclosed technology. This summary is not an extensive overview of the disclosed technology. It is intended neither to identify key or critical elements of the disclosed technology nor to delineate the scope of the disclosed technology. Its sole purpose is to present some concepts of the disclosed technology in a simplified form as a prelude to the more detailed description that is presented later.

In some aspects, example embodiments in accordance with the present disclosure can provide for adaptive cognitive training and combined music listening intervention, leveraging the advantages and techniques described herein. Also, example embodiments in accordance with the present disclosure can provide for cognitive training to treat cognitive dysfunction, including cognitive impairment.

In some aspects, the techniques described herein relate to a method for treating cognitive dysfunction, the method including: providing music to a user for a first time duration, and subsequently providing one or more cognitive training games to the user such that the user plays the one or more cognitive training games for up to a second time duration.

In some aspects, the techniques described herein relate to a system for treating cognitive dysfunction, including: one or more processors, and at least one memory including at least one non-transitory computer-readable medium storing instructions that, when executed by at least one of the one or more processors, cause the system to perform operations, the operations including: providing music to a user for a first time duration, and subsequently providing one or more cognitive training games to the user such that the user plays the one or more cognitive training games for up to a second time duration.

In some aspects, the techniques described herein relate to a non-transitory computer-readable medium for treating cognitive dysfunction, the non-transitory computer-readable medium storing instructions that, when executed by at least one processor of a computing system, cause the computing system to perform operations, the operations including: providing music to a user for a first time duration, and subsequently providing one or more cognitive training games to the user such that the user plays the one or more cognitive training games for up to a second time duration.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims or may be learned by the practice of such embodiments as set forth hereinafter.

### BRIEF DESCRIPTION OF THE DRAWINGS

To describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings.

FIG. 1 is a conceptual model of an example embodiment.

FIGS. 2A-2D are screenshots of example training games.

FIG. 3 is a graph of experimental results comparing cognitive training to a trivia question control.

FIG. 4 is a set of graphs of experimental results showing effects on non-trained working memory and verbal episodic memory.

FIG. 5 is a set of graphs of experimental results showing effects on spatial planning/reasoning and visuospatial working memory.

FIG. 6 is a set of graphs of experimental results showing effects on sleep.

FIG. 7 is a set of graphs of experimental results showing effects on insomnia.

FIGS. 8A-8E are graphs showing experimental results for a participant with insomnia and autism.

FIGS. 9A-9C are graphs of experimental results for quantification of neurodegeneration biomarkers in plasma.

FIG. 10 is a flowchart of an example method for treating insomnia.

FIG. 11 illustrates certain components that may be included within a computer system according to an example embodiment of the present disclosure.

Before explaining the disclosed embodiment of this disclosure in detail, it is to be understood that the invention is not limited in its application to the details of the particular arrangement shown, as the invention is capable of other embodiments. Example embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION

While the subject disclosure applies to embodiments in many different forms, there are shown in the drawings and will be described in detail herein specific embodiments with the understanding that the present disclosure is an example of the principles of the invention. It is not intended to limit the invention to the specific illustrated embodiments. The features of the invention disclosed herein in the description, drawings, and claims can be significant, both individually and in any desired combinations, for the operation of the invention in its various embodiments. Features from one embodiment can be used in other embodiments of the invention. In the description of the drawings, like reference numerals refer to like elements. The following description will provide a disclosure of various features, approaches, and aspects of example systems and methods that can overcome the limitations described above.

The present disclosure will now provide overview descriptions of various approaches to deploying embodiments of such systems and methods. It should be understood that the processes and algorithms described below are not limiting of the scope of this disclosure, can be combined in various configurations, and may be adapted to replace, complement, and/or fit with existing systems.

About 10-15% of those with mild cognitive impairment (MCI) progress to Alzheimer's Disease (AD) each year. MCI affects about 16% of older Americans, and about 45% experience comorbid insomnia, which further exacerbates cognitive symptoms. Both MCI and insomnia are associated with increased AD risk.

Insomnia symptoms are associated with cognitive disruption in healthy and cognitively impaired older adults. In older adults, insomnia is associated with worse verbal memory. Poor sleep quality and sleep efficiency are also associated with worse executive function, and both long and short sleep duration are associated with impairment across a range of tasks. Insomnia in older adults increases risk of MCI and AD. In older adults with MCI, greater time spent awake after sleep onset is associated with worse attention and executive functioning, and increased arousals are associated with worse nonverbal learning and problem solving. Prevalence of insomnia is approximately 45% in MCI2 compared to about 10-20% in healthy aging. Clearly, sleep and cognition are related, and insomnia is linked to cognitive decline. Therefore, there is a need to examine whether interventions aimed at improving cognition also improve sleep.

Insomnia symptoms are associated with increased pathology related to MCI and Alzheimer's Disease. Preclinical studies link insomnia to increased Alzheimer's related biomarkers (e.g., amyloid beta (Aβ) and tau), suggesting that sleep is essential for protein clearance and reduction of neuronal aggregations contributing to plaques and tangles (hallmark AD pathology). These studies suggest targeting insomnia may lower AD-related biomarker levels, thereby mitigating risk and delaying disease progression. Assessing the impact of cognitive training plus music listening on AD blood-based biomarkers may aid in understanding how cognitive training targets both cognition and sleep, and may promote future fully-powered investigations of neurodegenerative outcomes.

Sleep architecture may be a biomarker for AD pathology. As individuals age, there is a decrease in slow-wave sleep (e.g., “deep” or stage 3/N3 sleep) and rapid eye movement (REM) sleep. Slow-wave sleep has also been recently proposed as another potential biomarker of cognitive decline and impairment. For example, reduced slow wave activity during slow-wave sleep was found to be a mechanistic pathway through which amyloid beta pathology contributes to cognitive decline, especially memory consolidation, and Alzheimer's disease. Reduction in slow-wave sleep activity is also associated with a greater risk of cognitive impairment, and subsequent cognitive decline one year later is associated with reduced slow wave activity. Rodent work has found similar results, with reduced slow-wave sleep and sleep disturbances preceding cognitive deficit and AD neuropathology. Similarly, past work has found that individuals with dementia have reduced slow wave activity compared to controls, which has prompted the idea of slow-wave sleep as a promising intervention target for cognitive decline and Alzheimer's disease. Given that other insomnia treatments, such as cognitive behavioral therapy for insomnia (CBT-I), do not impact sleep architecture outcomes, studying interventions (e.g., cognitive training) that show promise for targeting not only behavioral sleep but also sleep architecture (e.g., slow-wave sleep) associated with AD is important and novel.

Blood-based biomarkers of Neurodegeneration/AD, such as Aβ (40/42) and tau, are cost-effective and less burdensome alternatives to measuring these biomarkers in cerebrospinal fluid. Although the concentration of these proteins is lower in blood plasma, examining them as an exploratory aim may be highly useful. Given the long pre-symptomatic stage in AD, where pathology may still be present, impact of promising interventions on this pathology may be assessed to inform early monitoring in a critical window prior to symptom onset.

Computerized cognitive training improves performance on non-trained tasks in older adults. Multiple studies show benefits of computerized cognitive training programs to non-trained cognitive tasks (e.g., transfer effects). Six weeks of cognitive training (e.g., using a Nintendo DS with a “Brain Age” software relative to a no contact control) improved working memory (e.g., digit span backwards; medium-large effects). Another study showed four-weeks of “Brain Age” improved processing speed and executive functioning. Another study found that five-weeks of “Brain Age” improved arithmetic and Stroop task performance. Generalization of effects, however, are hindered by a small number of studies with methodological limitations such as small samples, lack of customization, and consideration of adaptive training (e.g., titrated to individual performance to avoid stereotyped mode and unstable improvements)—important features of the multi-phased novel intervention approach of the present disclosure. It should be noted that, while cognitive transfer effects have been reported, other studies have found no evidence of such effects, prompting the need for more rigorous evaluation of cognitive training outcomes and potential pathways for cognitive training improvement (e.g., via music listening “boosters”).

Cognitive improvements after cognitive training are associated with cortical plasticity in insomnia regions. Cognitive training leads to increased activation in hippocampal and prefrontal cortex regions in healthy and cognitively impaired older adults. The prefrontal cortex and temporal cortex, are also altered in chronic insomnia. Therefore, targeting these regions through multimodal cognitive training, for example, that trains executive function (e.g., targeting prefrontal cortex), and memory (e.g., targeting the prefrontal cortex and hippocampus) is hypothesized to promote cognitive and neural activity in these regions and show enhanced effects on cognition and associated outcomes such as sleep.

Cognitive training is feasible in MCI and improves cognition. Older adults with lower overall cognitive scores and performance have been shown to benefit more (in comparison to those with better cognitive scores) from personalized multidomain cognitive training, thus suggesting that those with early cognitive deficits, such as MCI, may be an ideal group to test the range of benefit received from cognitive training. One study found that a multidomain computerized cognitive training (processing speed and working memory tasks; 100 minutes per day, 5 times per week for 6 weeks-50 total training hours) in individuals with MCI (M_{age}=74) was feasible (77% completed training) and resulted in trending improvement (p=0.09) in global cognitive function relative to an active control (passive computer activities). Similarly, another study found that a multimodal computerized cognitive training (processing speed, attention and memory tasks; 1-hour sessions, 2 times per week for 12 weeks-24 training hours) led to significantly greater improvements (ps<0.05) compared to an active control (computer activities such as trivia, category sorting, etc.)

Computerized cognitive training improves sleep in middle-aged and older adults. A recent study found 8-weeks of cognitive training reduced actigraphic sleep onset latency (medium effects) and wake time after sleep onset (medium effects), and improved sleep efficiency (medium effects), relative to an active ctrl that completed Microsoft Word/Paint exercises. Seven weeks of cognitive training (e.g., Medalia's Neuropsychological approach to mediation) plus psychoeducation improved sleep quality relative to a waitlist control. Another study showed 12 weeks of cognitive training (visual/auditory training) plus light stretching improved daytime sleepiness more than combined cognitive training/aerobic exercise. Although some prior cross-sectional survey analyses showed that greater volume of video gaming was associated with worse sleep quality in adults across the lifespan, they did not look at time of day of video game play. For example, video game play within 2 hours of bedtime may disturb sleep quality by increasing arousal prior to bed, which is why participants in the experimental trials did not complete cognitive training within 2 hours prior to bed, and did not separate video gaming into action-based violent games and non-violent/non-action-based cognitive training games. The latter has mainly showed associations with better sleep quality.

Slow-wave activity during sleep is enhanced through activities that promote brain metabolism during wake. Slow-wave sleep is known to be increased following activities that increase brain energy consumption during wakefulness. Cognitive training has been shown to increase regional blood flow to frontal brain regions, thus it is likely that this may lead to slow-wave sleep enhancement (e.g., increased proportion of slow-wave sleep relative to other lighter stages of sleep), and may serve as a mechanism underlying both cognitive transfer effects and behavioral sleep improvements (e.g., wake time after sleep onset, insomnia severity). Other studies have shown that slow-wave sleep enhancement (e.g., via pharmacological agents or electrostimulation) leads to better sleep continuity and improvement in insomnia symptoms (e.g., overall sleep quality and reduction in sleep onset latency (SOL) and wake after sleep onset, (WASO)).

Cognitive training shows promise for targeting physiological sleep and AD biomarkers. A recent study in younger adults showed that a multimodal cognitive training (e.g., Ruzzle word finding and decision making game) led to increased sleep continuity as evidenced by polysomnography (PSG) recordings. Of the limited evidence conducted, a recent review summarized the promising evidence regarding cognitive trainings impact on AD biomarkers (including electroencephalography (EEG), functional and structural neuroimaging, and blood-based biomarkers).

Listening to music improves cognition. Neuronal network priming is a proposed mechanism underlying how music listening can subsequently improve cognitive function. This states that music listening leads to neural firing patterns in distinct brain regions (e.g., a three-network neural model of creative cognition) that also subserve cognitive function. Thus, neurons are “primed” and the threshold for activation is reduced, facilitating cognition. A recent study showed music listening led to increased spectral power (whole brain beta, parietal theta, and whole-brain gamma) in default mode, cognitive control and salience networks. A meta analysis showed that music listening activation of functional brain regions may be more extensive than the three-network neural model, including multiple cortical, subcortical, and cerebellar regions that support attention and memory performance; these were not modulated by music training expertise. In support of these neurocognitive models of music listening, several studies have shown improvement in cognitive function following music listening in adults with and without cognitive impairment. A ten-week (90 minute sessions) leisure music listening intervention improved overall cognitive function and episodic memory in dementia patients. Studies that investigated the effect of music on patients with Alzheimer's disease (AD) also show that personalized song teaching sessions have a beneficial impact on the autobiographical memory. There is an association between music and memory retention in patients with AD, and older adults with AD perform better on a task of recognition memory for the lyrics of songs of a sung recording than a spoken recording. Even acute music listening (e.g., one 15-minute listening session) improved executive function and creativity in adults.

Music Listening improves insomnia symptoms in older adults. Music listening has been shown to offer some benefit to sleep for adults with primary insomnia. Some music interventions have been shown to reduce sleep onset latency and daytime dysfunction, and increase sleep duration and sleep efficiency in older adults. In one randomized controlled trial (RCT), relative to controls, individuals with insomnia listening to music at bedtime had better ratings for feeling rested, shortened stage-two sleep, and prolonged REM sleep. Another RCT in older adults showed that listening to music at sedative music tapes at bedtime for three weeks improved sleep quality, sleep duration, sleep efficiency, and reduced sleep latency, sleep disturbance, and daytime dysfunction.

Music listening interventions associated with changes to blood-based biomarkers of Alzheimer's disease. Similar to cognitive training, there is some preliminary evidence showing that music interventions also impact AD biomarkers. For instance, one study in older adults with subjective cognitive impairment showed that a classical music listening program was associated with less increase in blood plasma amyloid beta 42 (AβB42) relative to a meditation group. Example embodiments of the present disclosure may utilize music listening as a “booster” for cognitive training effects on AD biomarkers.

Example embodiments of the present disclosure may improve cognition, subjective everyday cognition, as well as behavioral sleep (for example, reduce insomnia severity and diary/actigraphy sleep onset latency, wake time after sleep onset), physiological sleep (e.g., increase the percentage of slow-wave sleep), and mood (e.g., anxiety, depression). Example embodiments may impact AD biomarkers, and may uncover potential mechanisms underlying efficacy of cognitive training.

Example embodiments provide a novel combined cognitive training that includes engaged music listening “boosters.” Example embodiments may provide a novel computerized combined at-home intervention that may allow for rapid translation outside of a clinical setting and into communities. Example embodiments may impact medicine, nursing, neurology, gerontology, and psychology. Example embodiments may provide a comprehensive assessment of sleep, mood, cognitive outcomes. Example embodiments may potentially uncover mechanisms by which cognitive training improves sleep/cognition. Example embodiments may provide an assessment of older adults with MCI and insomnia. MCI patients have difficulty with everyday cognitive function, thus, playing a computer game that requires minimal a priori effort or homework, such as that in other treatments such as cognitive behavioral therapy, may promote its use as a potential alternative or complimentary intervention that could be easily disseminated and may not require trained therapists to administer. Example embodiments may assist in exploration of the impact of cognitive training on blood-based AD biomarkers in MCI.

Example embodiments provide an adaptive cognitive training platform enhanced with music listening. Example embodiments may be provided to help underserved populations with mild cognitive impairment (MCI) and insomnia. Existing commercial cognitive training programs are generic, unengaging, and lack personalization or feedback. Example embodiments integrate tailored gameplay with music-based cognitive priming to address both sleep and cognition. Furthermore, existing programs may offer limited scope for ongoing refinements based on in-depth user feedback, reducing their potential efficacy. Consequently, there remains a notable gap in the development of targeted, flexible, and user-informed solutions for improving cognition and sleep in these vulnerable groups, which is addressed by example embodiments of the present disclosure.

Cognitive training programs have proliferated in recent years, yet many rely on static difficulty levels, generalized content, and commercial designs that limit adaptability and access. Example embodiments provide a user-centered, multimodal cognitive training platform that stands apart through its computerized titration of task difficulty, dynamically adjusting content based on each user's performance and feedback. Unlike commercial tools, which offer generalized cognitive exercises primarily for healthy adults, example embodiments may be purposefully designed for clinically underserved populations, including older adults with insomnia and autistic adults. Thus, example embodiments may be utilized to treat users (e.g., patients) who have been diagnosed with insomnia and/or autism.

Consistent with the principle of neuroplasticity and the Capacity Efficiency Cognitive Training Model, multimodal commercialized cognitive training leads to greater improvement relative to controls in cognition and associated functions such as sleep and mood in older adults with insomnia who are cognitively healthy or have MCI. Experimental results also suggest that, in MCI patients with insomnia, cognitive training-related cognitive improvement (e.g., near cognitive transfer effects) is associated with physiological sleep changes, such as increased amount of slow-wave sleep (SWS)). These are exciting findings given the lack of trained providers for other behavioral sleep interventions and the known sleep medication adverse side effects, such as increased falls and cognitive disturbance. Example embodiments of the disclosed technique provide a novel cognitive training program (referred to herein as “COGMUSE℠” cognitive training components) and have evaluated its usability in older adults with insomnia. Other behavioral interventions, such as active music listening, have also been shown to improve cognition in AD patients, and improve sleep in older adults (e.g., reduce sleep onset latency and daytime dysfunction, and increase sleep duration and sleep efficiency). This is due to music's impact on structural and functional neuroplasticity in regions associated with attention, reward processing, and executive function. However, no one has evaluated whether music listening provides additional benefit to cognitive training across both near transfer (e.g., cognition) and far transfer (e.g., sleep and mood) outcomes. Further, no one has tested the potential mechanisms (e.g., physiological sleep, neuroplasticity) contributing to these transfer effects. Example embodiments add music listening to cognitive training. Systematic evaluation of a novel cognitive training plus music listening intervention will allow for easy implementation of test scenarios, monitoring, and iterative optimization.

Older adults with MCI and insomnia experience not only cognitive disruption, but their poor sleep exacerbates this disruption. Example embodiment provide a novel adaptive cognitive training plus music listening intervention (“COGMUSE℠”). In older adults with MCI and insomnia, relative to cognitive training alone or an active control (e.g., online trivia assignments), example embodiments lead to greater improvement in cognition, behavioral sleep and mood, via mechanistic pathways of sleep physiology and neuroplasticity. Example embodiments provide a mixed-methods multi-phased (or multimodal) approach. Example embodiments may affect primary near transfer (e.g., cognition) and secondary far transfer behavioral outcomes (e.g., sleep and mood) in older adults with MCI and insomnia.

Example embodiment provide a multimodal adaptive cognitive and music engagement intervention, which may provide a greater benefit to cognition (e.g., computerized task performance), behavioral sleep (e.g., sleep onset latency, wake time after sleep onset, total sleep time) and mood (e.g., anxiety and depressive symptoms), as compared to cognitive training alone or active control. Example embodiments may affect secondary mechanistic outcomes, e.g., physiological sleep and neural structure/function, in older adults with MCI and insomnia.

Example embodiments provide greater benefit to physiological sleep (e.g., percent of slow-wave sleep (SWS)) and neural outcomes (e.g., frontal/temporal gray matter volume, fronto-temporal connectivity) in comparison to cognitive training alone or active control.

Example embodiments leverage a relationship between behavioral and mechanistic outcomes, and their potential moderators, e.g., game engagement, adherence, music preferences, and cognitive reserve. Example embodiments may provide for cognitive training to treat cognitive dysfunction, including cognitive impairment.

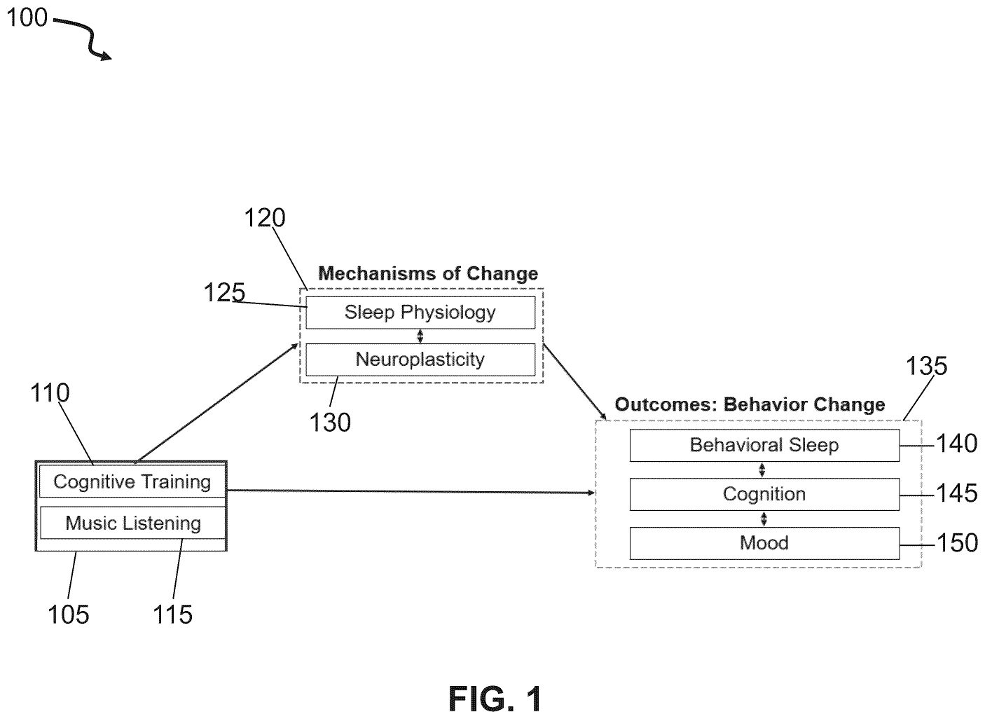

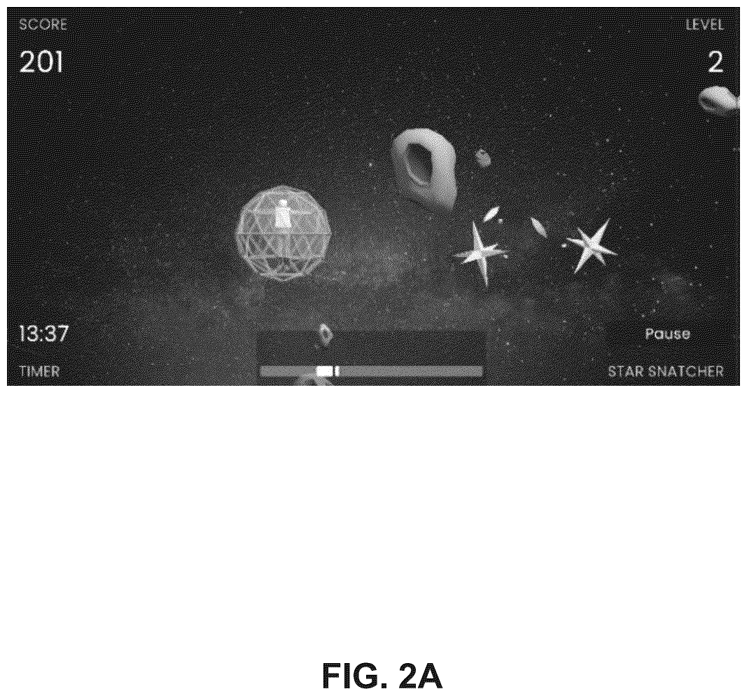

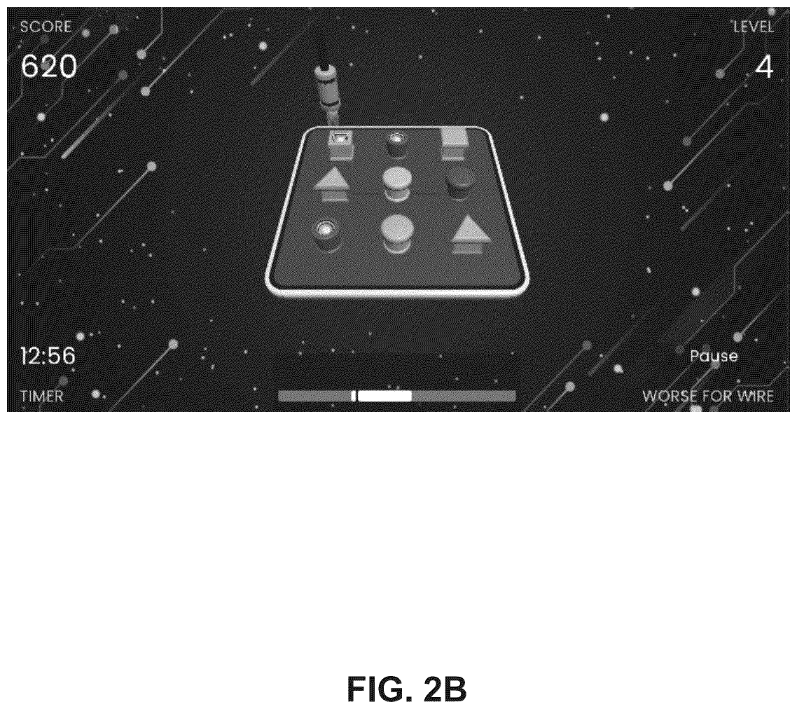

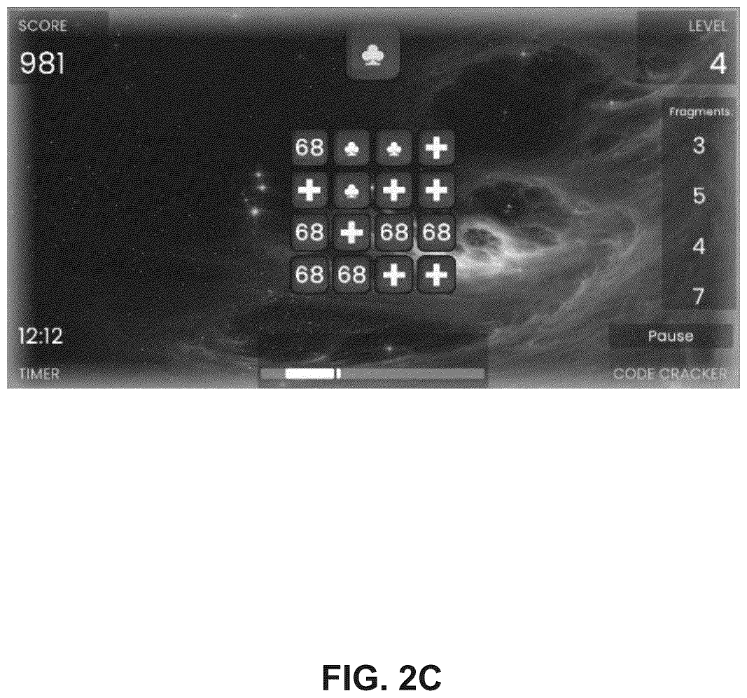

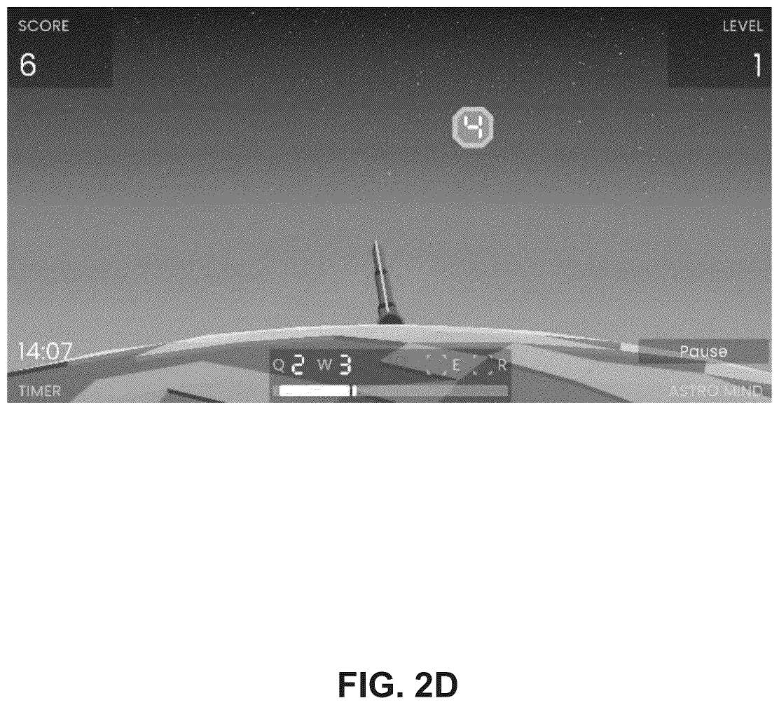

FIG. 1 is a conceptual model of an example embodiment. FIGS. 2A-2D are screenshots of example training games.

As illustrated in the conceptual model 100 of FIG. 1, example embodiments may provide a training system 105 that provides cognitive training 110 combined with music listening 115. The training system 105 drives mechanisms of change 120, which include sleep physiology 125 and neuroplasticity 130. Both the training system 105 and the mechanisms of change 120 affect patient outcomes 135, including behavioral change, which includes behavioral sleep 140, cognition 145, and mood 150.

Example embodiments may incorporate one or more training games, each developed to train on one or more targeted skills. In the example used in experiments, four training games were provided:
“Star Snatcher” is shown in FIG. 2A, and provides training for processing speed, attention, perceptual-motor skill, and inhibition;
“Worse for Wire” is shown in FIG. 2B, and provides training for processing speed, attention, visuospatial working memory, and inhibition;
“Code Cracker” is shown in FIG. 2C, and provides training for processing speed, attention, visuospatial working memory, and inhibition; and
“AstroMind” is shown in FIG. 2D, and provides training for spatial attention, perceptual-motor skills, processing speed, and inhibition.

A music engagement “booster” task precedes each training/therapy session within an example interface. The booster enhances cognitive readiness and potential training/therapy effects. For example, the music engagement booster may have a 15-minute duration, although embodiments are not limited thereto. Certain types of music can prime specific areas of the brain. Example embodiments may provide music that activates the same brain areas that are targeted by the cognitive training games, for example, to “boost” the specific training goal(s).

The system may program (or “prescribe”) specific games or music booster to each user based on the user's cognitive profile. For example, a program of which training game(s) should be used, the difficulty level of the game(s), and/or the music booster type and/or length may be personalized to each user. Such personalization may be provided by a therapist or other clinical expert, or may be provided automatically using machine learning or artificial intelligence techniques, for example, to iteratively adjust the prescriptions. The cognitive training games may be adjusted to have visual and auditory elements, instructions, rewards, and timing to cater to the specific needs of each patient, including older adults and autistic adults.

Example embodiments may provide a simplified user-friendly instructions tested and developed for older adults. For example, graphics may take into account feature and/or design preferences that are common for older adults. Example embodiments may provide a simpler interface than other systems. For instance, example embodiments may use a mouse-based interface. Some example embodiments may avoid use of a joystick or a keyboard, for example, depending on the requirements of a user (e.g., a patient). Some example embodiments may use a touch screen interface. As another example, simplified visual plus text instructions may be provided. Example embodiments may provide feedback at the end of each game session or after each segment of the game session. For example, each game session may be provided in 15-minute segments, with feedback provided after each segment. This is only an example, and embodiments are not limited to 15-minute sessions or segments. Example embodiments provide a music listening component prior to cognitive training, which is not found in conventional cognitive training methods. Example embodiments may provide multimodal cognitive training targeting and training for multiple cognitive domains. Example embodiments may provide iteratively adaptive training over time. As a nonlimiting example, training may be provided daily for up to 6 weeks. Example embodiments may provide iterative development, for example, to increase or maximize instruction uptake.

Example embodiments may utilize a user-centered design approach that considers age-related cognitive/perceptual concerns in a target population. Example embodiments may support older adult readability, for example, sans serif typeface, large font (e.g., 14 points or larger), clear content headings, non-cluttered backgrounds, brief videos, bright colors, visual contrast, representative icons, content enhancing, non-distracting audio, clear navigation, and/or limited pull-down menus. Example embodiments may promote MCI participant use, for example, using simplified language, visuals, and/or guidance.

### EXAMPLE IMPLEMENTATIONS AND EXPERIMENTS

The following is a description of the methods for experimental implementation of example embodiments of the present disclosure and some results of experimental testing.

### I. Participants and Approach

### A. Stage 1: Initial Testing

Stage 1 includes small focus groups and usability testing, generating qualitative and quantitative feedback.

### B. Stage 2: Pilot Trial

Thirty-four (34) participants with MCI and insomnia (see inclusion criteria) will be recruited. Participants will be randomized to complete either a 6-week cognitive training that includes music listening boosters (COGMUSE℠; n=17) or only the cognitive training component of COGMUSE℠ without music listening (n=17). Baseline and post-intervention assessment period duration will be two weeks (to account for daily sleep diary and actigraphy assessments). At baseline and post-intervention, participants will complete measures of cognition, mood, and sleep. In a subsample (n=15) of participants, the preliminary impact of COGMUSE℠ was explored on blood-based AD biomarkers (tau & AβB42). To account for about 15-20% attrition, 41 participants may be recruited for Stage 2 to reach a final pilot sample of 34. The sample is representative of the diverse Tampa, Florida (FL) area. Florida is tied with Georgia as having the nation's ninth-most diverse population, with a diversity index of 64.1%. Florida also has the sixth-highest Hispanic population among states, accounting for 26.5% of Florida's residents. Hillsborough County, FL is the third most diverse county in the state with a diversity index of 67.8%. Of the 1.47 million people in Hillsborough County, FL, 47.3% are white (non-Hispanic), 22.3% are white (Hispanic), 15.9% are Black, and 4.49% are Other (Hispanic).

### C. Stage 1 and 2 MCI Participant Inclusion/Exclusion Criteria

### (1) Inclusion Criteria

(a) 60+ yrs of age, (b) meet criteria for MCI based on based on established criteria and will include: (i) subjective memory complaint, (ii) Montreal cognitive Assessment (MoCA)—MoCA score falls between 18/30 and 25/30, (iii) Clinical Dementia Rating (CDR) memory box score is 0.5-total CDR of 0.5, (iv) intact activities of daily living (ADLs), and (v) participants scoring 1.5 standard deviations below normative values (based on age and education) on objective memory task (National Institutes of Health (NIH) Toolbox working memory or verbal task). NIH Toolbox provides normative T-scores for easy MCI cutoff calculation; (c) no neurological or psychiatric illness or dementia, (d) non-gamers (report <2 hours of video games per week over the last 2 yrs, considered “casual gamer”) b, (e) proficient in English (reading and writing), (f) meet clinical diagnosis criteria for Insomnia: (i) insomnia complaints for 6+ months that (ii) occur despite adequate opportunity and circumstances for sleep, (iii) consist of 1+ of the following: difficulty falling asleep, staying asleep, or waking up too early, (iv) daytime dysfunction (mood, cognitive, social, occupational) due to insomnia.

### (2) Exclusion Criteria

(a) Unable to provide informed consent, (b) unable to undergo randomization, (c) other sleep disorder (sleep apnea [apnea/hypopnea index, AHI>15], Periodic Limb Movement Disorder-PLMD [myoclonus arousals per hour>15]), (d) shift-worker, (e) significant medical disorder (e.g., cancer), (f) severe untreated psychiatric comorbidity that renders randomization unethical, (g) other major psychopathology, except for depression or anxiety (e.g., suicidal ideation/intent, psychosis), (h) psychotropic or other medications (e.g., beta-blockers) that alter sleep, (i) uncorrected visual/auditory impairments, (j) participation in nonpharmacological treatment for sleep/fatigue/mood/cognition outside the current study, (k) less than 3 years of formal music training and not currently engaged in formal music training (confirmed by global score on Goldsmiths Musical Sophistication Index (Gold-MSI)). Volunteers may be familiar with gaming and show less room for improvement (selection bias), but concern is mitigated because participants must report <2 hours of videogaming/week, and gaming hours are examined as potential covariate/moderator of treatment effects. Only shift-work is exclusionary. Employment status will be recorded and examined as potential covariate in analyses. Medications impacting sleep or cognition represent confounds. However, once stabilized for 6+ weeks, participants on sleep medications are eligible as insomnia symptoms tend to return to baseline, reducing confound. Sleep and MCI medications (e.g., Aricept, Aduhelm, etc.) use may be recorded on sleep diaries and examined statistically.

### D. Polysomnography (PSG; 2 Overnights)

Two nights of PSG (1 adaptation night, 1 recording night) will rule out sleep disorders other than insomnia (e.g., apnea), and provide PSG outcomes. Participants will sleep in their own beds. The SLEEP PROFILER™ System (Advanced Brain Monitoring) will be used. This device is self-applied, with a three-frontopolar electroencephalography (EEG) acquired from a flexible headband (using disposable wet electrodes, airflow using a nasal cannula and pressure transducer, head movement/position by actigraphy, snoring with acoustic microphone, pulse from the forehead/finger, wireless wrist oximetry, and thorax/abdomen respiratory effort). Participants are also provided with a video of how to complete the setup in their home. There was high agreement for between SLEEP PROFILER™, diaries, and total sleep time (TST) on behavioral sleep metrics (total sleep time, sleep efficiency, wake time after sleep onset and sleep onset latency). The SLEEP PROFILER™ system provides excellent estimation of slow-wave sleep and REM sleep in adults. The SLEEP PROFILER™ has been tested and well tolerated (completed at three timepoints for 95% of older adult participants in a recently completed clinical trial and one other 2-week study in older adults with insomnia.

E. Sleep Diary Confirmation of Insomnia (about 5 Mins/Day)

Baseline sleep diaries will confirm insomnia diagnosis and must show: >30 minutes of sleep onset latency or wake time after sleep onset on 6+ nights/14 during baseline. Ambulatory monitoring is more sensitive than inpatient monitoring for distinguishing insomnia from normal sleepers, and is comparable to inpatient PSG for apnea detection, reduces inconvenience, and increases acceptance.

### II. Development

### A. Initial Development

Iterative game development and optimization was applied to develop the systems and methods in accordance with the present disclosure, referred to as “COGMUSE℠” Example embodiments (e.g., COGMUSE℠) include games that activate, exercise, and strengthen cognitive processes, for example, attention, memory, executive functions, and/or processing speed. In some experimental setups, each game had a duration of about 15 minutes. In other experimental setups, each game had a duration of about 20 minutes. Similar to other cognitive training programs, such as COGNIFIT®, the games may be modeled after existing neuropsychological tasks. Target training domains were selected based on secondary analyses showing multi-domain attention, processing speed, and executive function cognitive training associated with non-trained cognitive improvement in older adults with and without cognitive impairment. The “Star Snatcher” game (see FIG. 2A) targets spatial attention (e.g., a user should pay attention to targets on screen), inhibition (e.g., a user should ignore and/or doge non-targets), perceptual motor skill (e.g., a user should move an avatar to a target with a mouse), and processing speed (e.g., a user should move quickly). The “Worse for Wire” game (see FIG. 2B) targets spatial attention (e.g., a user should pay attention to colored targets), inhibition (e.g., a user should ignore non-target shapes and colors), and processing speed (e.g., a user should move quickly). Qualitative data from commercialized COGNIFIT® cognitive training and COGMUSE℠ initial cognitive training cognition was tested in older adults with and without MCI, and additional optimizations were made. All activities had a speeded component and increase in difficulty as a player advances throughout the game. Thus, games were adaptive and titrated to individual performance and become more difficult as the player progresses, e.g., to maintain engagement and maximize benefit. Additional and/or other cognitive training games are within the scope of embodiments of the present disclosure. The game features have been refined to be capable of estimating the cognitive abilities of the patients through the play sessions and may, for example, provide participants with an overall “brain score.” Experiments provided appropriate software and hardware (e.g., keyboard, mouse, touchscreen).

### (1) Game Development

Game art models were produced using industry standard modeling software, including Blender and Maya. Models are then exported to Unity 3D, a cross-platform game engine to create the interactive game environment. Games will be hosted on a server and accessed by users through a web browser using iPad tablets. User data (play duration, date and time, scores) from game play will be transferred to and stored in a database for researchers to retrieve and analyze.

### (2) Music Listening Component

In the experiments using the disclosed technique, a 15-minute music listening session was administered prior to a user's playing the cognitive training games. This music listening component fosters attention to patterns (e.g., formal musical structures, timbres, sequences, etc.) that will prepare the user for visual and auditory functioning in the COGMUSE℠ program. Active music listening relies upon neural networks that support attention as active listening bilaterally activates multiple cortical, subcortical, and cerebellar regions including six functional brain networks. Most music listening studies in older adults and clinical populations include classical music as the focus due to the complex patterns that stimulate cognitive performance. Participants learn about the elements of music during short 15-minute segments prior to engaging with the COGMUSE℠ program. Pieces, chosen based upon pattern absorption potential, were of the classical and folk genre (no lyrics). Attention-based responses are requested during the listening session (e.g., press the space bar when you hear a return to the main trumpet theme in the Haydn Concerto for Trumpet in E Flat, Third Movement).

### B. Pilot

Participants completed the intervention for 60 minutes per day, 3 times per week for 6 weeks at home for 18 hours total. Participants were randomized to (1) music listening 15-minute “boosters” within web-based training program (n=17) or (2) cognitive training only, with no music listening (n=17). To be consistent with sleep hygiene recommendations, participants were instructed to not complete intervention within 2 hours of bedtime. A report provides activities played and highest score achieved. Pilot data showed that these techniques are feasible with good intervention adherence.

### III. Measures

### A. Cognition

### (1) Objective Cognition—National Institutes of Health Toolbox—Cognition Battery

A computerized cognitive test battery measuring performance across several domains: executive function (Flanker inhibitory control task-using computed score that take into account accuracy and reaction time; Dimensional Card Sort Test, measures cognitive flexibility, using accuracy score), working memory (List Sorting Task, total number of words recalled across list 1 and 2), verbal (episodic) memory (Auditory Verbal Learning Task, number of words recalled across learning trials 1-3 and delayed recall trial to measure long-term episodic memory) will be administered. It has very good test-retest reliability and strong construct and convergent validity with other widely used neuropsychological test measures.

### (2) Stroop Color-Word Test

Programmed and presented using software to provide indices of processing speed (reaction time-control trials), processing speed and attention (reaction time-congruent trials) and inhibition (executive function, reaction time-incongruent trials). Stroop was chosen because it is sensitive to change over time and its potential utility as an early marker of Alzheimer's disease.

### (3) Subjective Everyday Cognition

The Patient-Reported Outcomes Measurement Information System Cognitive Function (PROMIS-CF; from the NIH Toolbox V3) is a six-item questionnaire about perceived cognitive deficits. Participants are asked to identify how often they have experienced cognitive deficits in the past 7 days from “never” (score=5) to “very often/several times a day” (score=1). PROMIS-CF has been found to be associated with the Montreal Cognitive Assessment (MoCA) scores in older adults, and 1-year decline in PROMIS-CF scores were associated with new cognitive impairment diagnoses.

### B. Mood

### (1) Geriatric Depression Scale (GDS; 15-Item Short Form)

The GDS short-form will be used to assess depression. This assessment was chosen due to some other depression scales containing questions regarding somatic symptoms that may be due to other age-related comorbidities rather than depressed mood. A 15-item inventory asks respondents to endorse no (no depressive feelings) or yes (experience depressive feelings) regarding their feelings towards various aspects of daily living/situations. Higher total scores indicate worse depressive symptoms. A cutoff score of 5 suggests presence of depression. The GDS has excellent psychometric properties in healthy older adults and those with mild cognitive impairment.

### (2) State-Trait Anxiety Inventory (STAI-Y1)

An inventory that asks respondents to rate how true twenty self-descriptive statements (e.g., “I feel calm”) are on a 4-point scale (1=not at all, 4=very much so). Typically, respondents are asked to rate statements according to how they generally feel (trait-anxiety scale) and how they feel in the current moment (state-anxiety scale). Total scores range from 20 to 80, with higher scores indicating greater maladjustment. The STAI-Y1 has good psychometric properties in older adult populations.

### C. Behavioral Sleep

### (1) Subjective Behavioral Sleep

Insomnia Severity Index (ISI) 127; Electronic Daily Sleep Diaries. Online diaries completed each morning (about 5 mins) during 2 week assessment periods. Variables: sleep onset latency (time from lights-out until sleep onset), wake time after sleep onset, total sleep time. Variables chosen due to most change in pilot data and common sleep complaints in insomnia diagnosis criteria.

### (2) Objective Behavioral Sleep-Daily Actigraphy

Actigraphy Protocol. GENEACTIV® (ACTIVINSIGHTS®) is a watch-like device that monitors light and gross motor activity, will be worn 24/7 for 2 weeks at each assessment point. Accelerometers are set to sample at a frequency of 87.5 Hz, with data storing in 5-second epochs. Data is analyzed by open-source data analytic tool R using the GGIR package. GGIR was developed for GENEACTIV® accelerometers and uses raw acceleration ENMONZ values (Euclidian norm minus one with negative values set to zero). For sleep metrics, GGIR identifies periods of sustained inactivity where there is a smaller change in arm angle than a predefined threshold. Threshold parameters are defined as a change in arm angle of five degrees over a five-minute period. These thresholds have shown good accuracy for sleep detection compared to PSG. Use of GENEACTIV® and data analysis using GGIR for older adults has been validated. 132-135 Past work has indicated GENEACTIV® performed similarly to ACTIWATCH®-2, with no significant inter-device differences for any sleep parameters. To reduce the burden for MCI patients, participants wear device the 24 hours a day, 7 days a week during each 2-week assessment period. Actigraphy will measure same sleep outcomes as diaries.

### D. Physiological Sleep

Objective Physiological Sleep-PSG measured sleep stages. Two-night (1 adaptation night and 1 recording night) PSG measured with SLEEP PROFILER™ at each timepoint (within first 2 weeks of baseline, post-treatment). PSG records are scored by SLEEP PROFILER™ proprietary software according to American Academy of Sleep Medicine (AASM) criteria. The PSG data was stored on a secure Health Insurance Portability and Accountability Act (HIPPA)-compliant portal that includes enterprise grade Secure Sockets Layer (SSL) encryption with automatic cloud backups with every subscription plan to keep data secure/safe. PSG records provide a percent of time in slow-wave sleep (stage 3/N3).

### E. Biomarkers of Neurodegeneration

Neurodegenerative biomarkers-tau and AβB42. Blood samples are collected from fasting participants between 9-11 a.m. and stored in a −70° freezer within the lab. The MRM (multiple reaction monitoring) approach is used for the non-protein samples. The lab has a sensitive triple quadrupole mass spectrometer coupled with an ultrahigh performance liquid chromatography system that is ideal for these measurements. For protein measurements, two approaches were used: quantitative spectral counting of intact proteins and measuring the peptides from the digested proteins. Peptides matching: the proteins will be mapped using software. Initial list of peptides from the software will be honed using trypsin digests of commercially-available purified proteins and peptides. Commercially-sourced proteins and peptides will be authenticated by mass spectrometry, to confirm sequence of individual trypsin-derived peptides. Synthetic peptides will aid in quantitation of the two tau phospho-site variants.

### F. Game-Related Experience

Sessions played: GRA checks adherence though an online portal at twice-weekly check-ins. Participants also provide qualitative feedback on the games played. Weekly game scores: Maximum test score recorded. Game Engagement Questionnaire-core module: Participants rate from 0 (“not at all”) to 4 (“extremely”) the degree to which they agree with 33 aspects of game experiences. The total score provides a measure of game engagement. Music Engagement: Based on previously-described methods.

### G. Covariates Related to Music Listening and Preferences Outside Study

Music Engagement & Preferences: Based on other music protocols, participant music preferences and music engagement are evaluated via the Assessment of Personal Music Preferences (APMP), and examined as a potential covariate in analyses. Also assessed was daily music listening (e.g., number of minutes of listening to music via radio, TV, web, smartphone, etc., or minutes of musical instrument playing and/or singing in a band, choir, chorus, or solo) in the afternoon/evening diaries, and examine as a covariate.

### H. Covariates Related to Etiology of Insomnia

The etiology insomnia in MCI is likely multifactorial, therefore contributing factors are measured within existing assessments, including socio-demographics and multi-morbidities (e.g., during a clinical interview), lifestyle-related factors such as physical activity (e.g., daily afternoon or evening diaries using brief modified version of the Godin-Shepherd Leisure-Time Exercise Questionnaire), and psychological factors (e.g., depression and anxiety, which are already secondary outcomes). The contribution of these factors as covariates may be assessed in analyses and future secondary analyses as moderating factors.

### IV. Data

The data was examined by persons with have experience in behavioral intervention optimization and cognitive training development. Four clinical trials have been conducted on digital and web-based cognitive training in aging populations (cognitively healthy older adults, older adults with MCI, middle-aged adults with generalized anxiety disorder) with insomnia disorder. Development of the disclosed technique have been worked on using on scalable web-based behavioral intervention translation and dissemination. Two recent trials were conducted developing a music engagement intervention an in-music listening engagement and combined cognitive training and music training.

Computerized cognitive tasks/training in older adults. Experiments showed that older adults can use environmental cues of various modalities (e.g., visual, auditory, tactile) to improve computerized working memory performance, showing similar improvement as younger adults. When the working memory task was more difficult (e.g., participants performed a decision making task during memory maintenance), older adults showed greater benefit from cues than younger adults. These studies were also important for establishing that older adults can perform novel computerized tasks and benefit from environmental support to improve cognition.

Better sleep is associated with better cognition in older adults. Better self-reported and objectively assessed (via actigraphy) sleep are associated with better performance on tasks measuring executive functioning and memory in older adults with insomnia. These results provide support for including sleep and cognition in the model for example embodiments and suggest bidirectional associations (e.g., improving cognition may improve sleep).

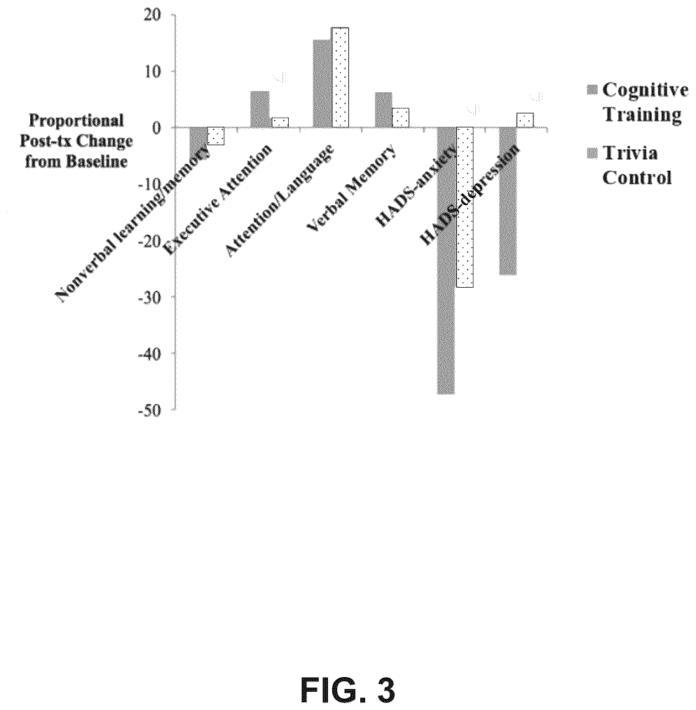

FIG. 3 is a graph of experimental results comparing cognitive training to a trivia question control.

Cognitive training leads to transfer effects on cognition and associated functions in cognitively healthy and cognitively impaired aging adults. Middle-aged adults-Cognitively Healthy. In the pilot experiment (n=14) evaluating cognitive training (Nintendo DS “Big Brain Academy”), executive functioning improved (with a large effect) after 6 weeks of cognitive training (but not with trivia question control; see FIG. 3). As shown in FIG. 3, proportional post-treatment changes from baseline were measured in six areas: nonverbal learning/memory, executive attention, attention/language, verbal memory, HADS-anxiety, and HADS-depression. As can be seen, there were significant differences (p<0.05) between cognitive training and the control.

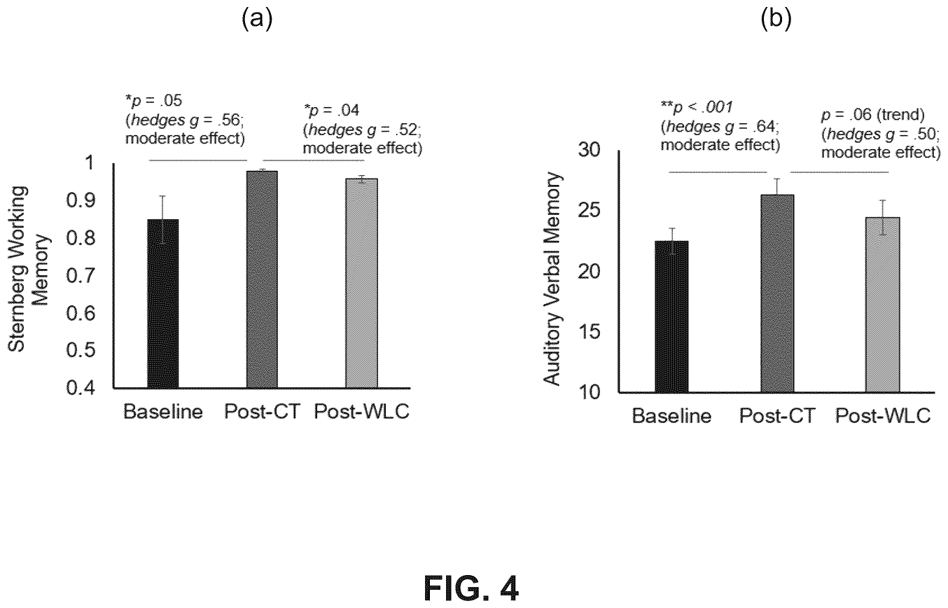

FIG. 4 is a set of graphs of experimental results showing effects on non-trained working memory and verbal episodic memory.

Cognitively Healthy Older Adults with Insomnia. Six weeks of cognitive training (“Big Brain Academy”; 18 hours; see FIG. 4) improved cognition more than control (cross-over design). As shown in graph (a) of FIG. 4, Sternberg working memory trended higher than baseline after cognitive training (moderate effect), but scores did not differ following waitlist control (WLC) (p=0.11). Sternberg scores following cognitive training (Post-CT) were also higher than WLC (moderate effect). Graph (b) of FIG. 4 shows that Auditory Verbal Memory was better than baseline at Post-CT (moderate effect), but scores did not differ from baseline at Post-WLC (p=0.07). Post-CT auditory verbal memory scores were also trending towards being statistically significantly higher at Post-CT versus Post-WLC (moderate effect).

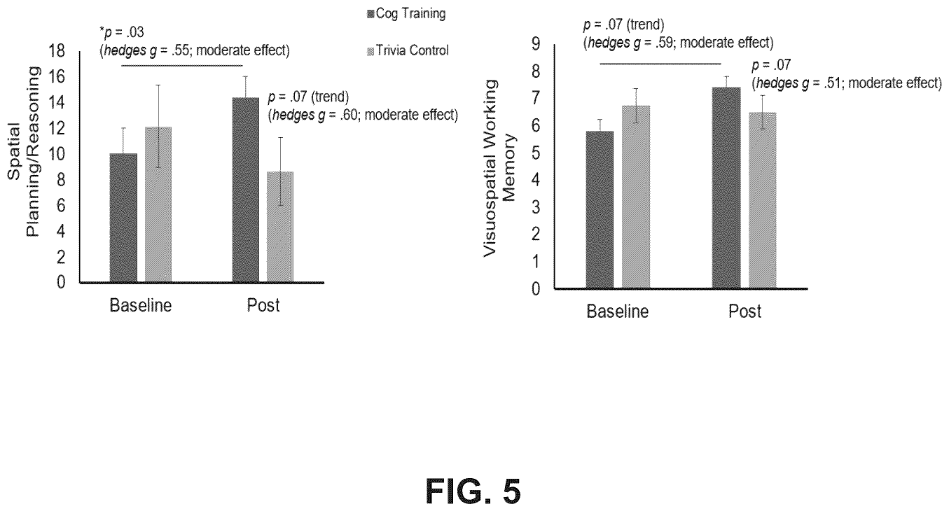

FIG. 5 is a set of graphs of experimental results showing effects on spatial planning/reasoning and visuospatial working memory.

Cognitive training is feasible in MCI and insomnia, well tolerated and enjoyable, and improves cognition more than active control. A pilot experiment showed 6-week cognitive training (n=19; 60 minutes, 3 times/week for 6 weeks=18 hours) was feasible (average completion rate of 94%, range from 79-100% completion), well tolerated, and enjoyable. Additionally, as shown in FIG. 5, cognitive training (n=19) improved cognitive function in spatial planning/reasoning (executive function, see graph (a) of FIG. 5) and visuospatial working memory (see graph (a) of FIG. 5) with moderate significant and/or trending effects more than the active control group (n=8; 2:1 assignment to test feasibility and initial effects) who completed trivia questions online (see FIG. 5, graphs (a) and (b)).

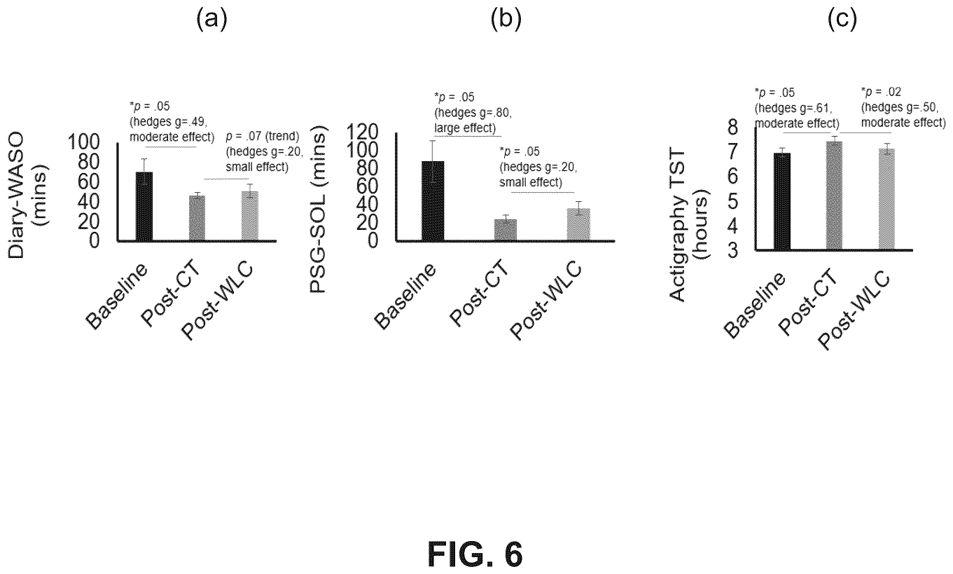

FIG. 6 is a set of graphs of experimental results showing effects on sleep.

Cognitive training improves sleep more than control in cognitively healthy and MCI older adults with insomnia. In an experiment with cognitively healthy older adults with insomnia, data n=24; see FIG. 6) showed 18 hours of cognitive training (using “Big Brain Academy”) led to greater improvement than waitlist control (WLC; crossover design) in diary wake time after sleep onset (small-moderate effects, see graph (a) of FIG. 6). PSG sleep onset latency (SOL; small-large effects, see graph (b) of FIG. 6) and actigraphy total sleep time (moderate effects, see graph (c) of FIG. 6).

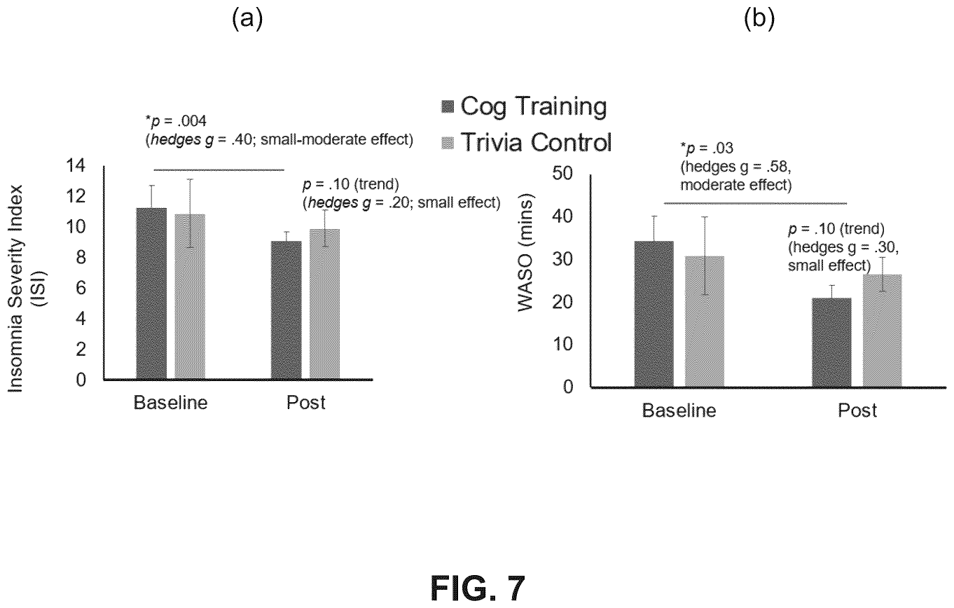

FIG. 7 is a set of graphs of experimental results showing effects on insomnia.

Older adults with MCI and insomnia. Experimental results showed cognitive training (18 hour, 60 minutes, 3 times/week for 6 weeks) improved insomnia severity (see graph (a) of FIG. 7) and wake time after sleep onset (see graph (b) of FIG. 7) to a greater degree than active control, which was only significant within group change for cognitive training, not trivia questions—with moderate effects; trending group differences at post-intervention had a small effect. Importantly, this pilot data shows that remote at home cognitive training targets sleep outcomes of interest in MCI participants.

Improvement in Cognition after Cognitive Training associated with reduced slow-wave sleep (SWS) in MCI patients. Experimental data from MCI patients with insomnia (n=13) shows that following cognitive training (3 hours/week for 8 weeks), there is a trend showing improvement in non-trained cognitive tasks is associated with increased amount of slow-wave sleep. Comparing post-cognitive training difference scores, improvement in the Paired Associates task (Cambridge Brain Sciences battery, which measures verbal memory) was positively correlated (=0.64, p=0.06) with amount of post-intervention increase in slow-wave sleep.

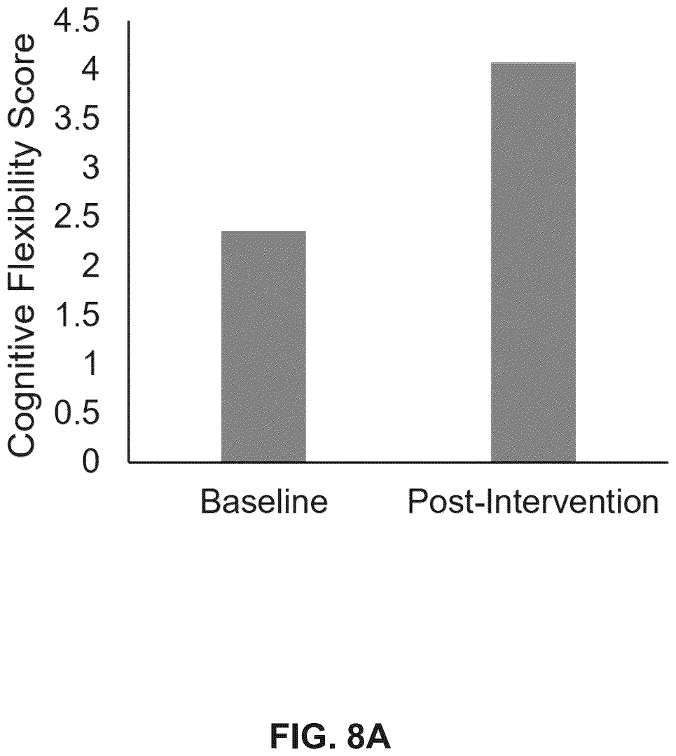

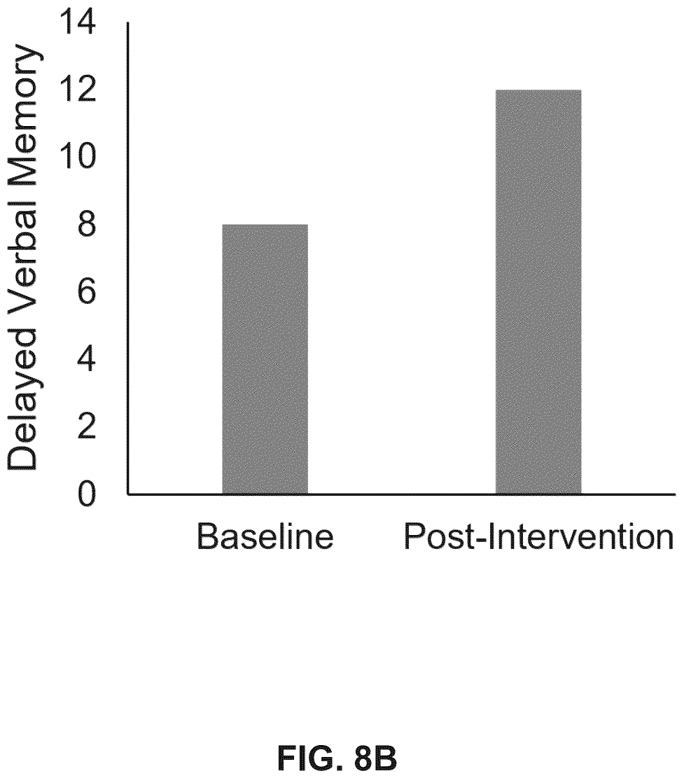

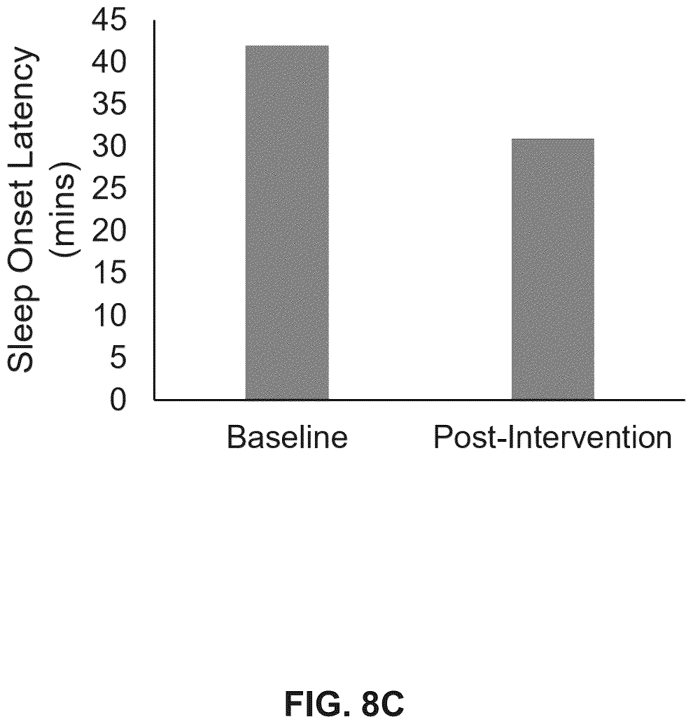

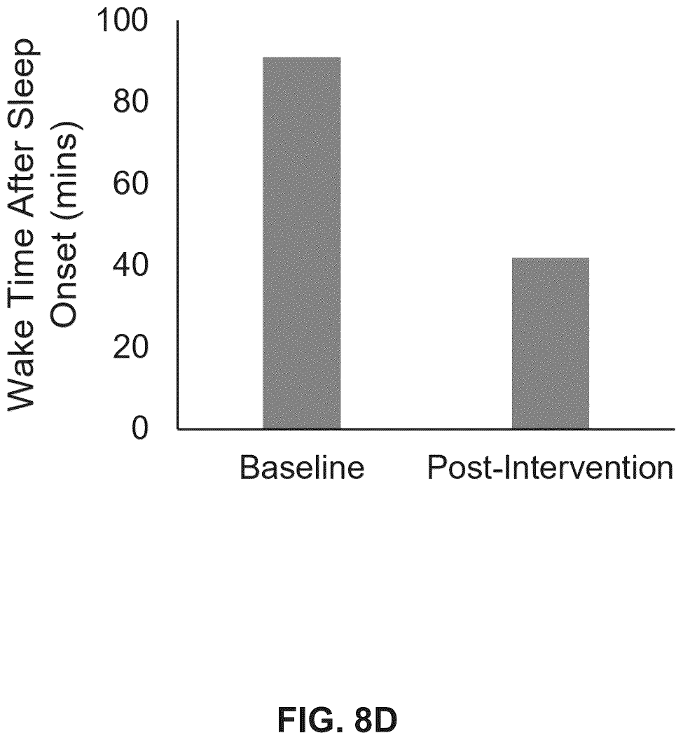

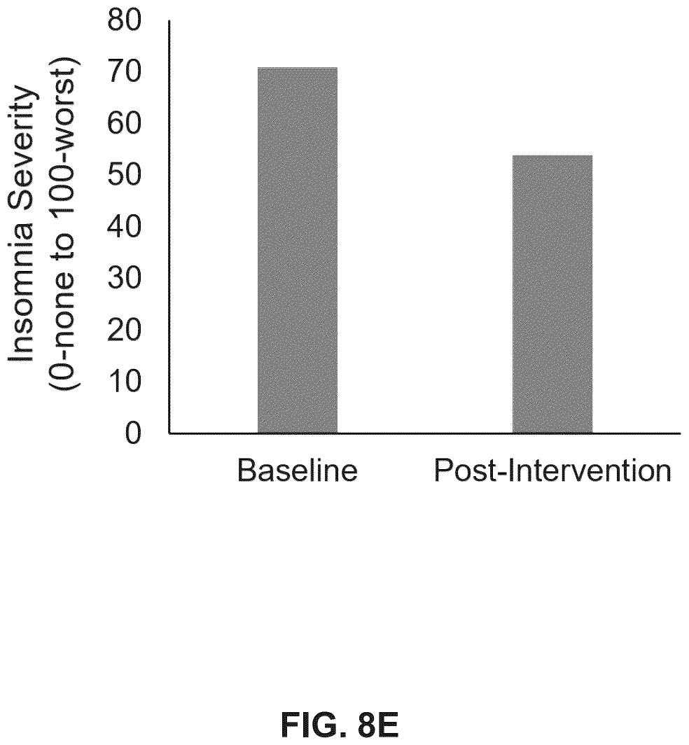

FIGS. 8A-8E are graphs showing experimental results for a participant with insomnia and autism.

Experiments using the disclosed technique were run combining cognitive training with a music booster for several autistic adults with insomnia through the pilot trial. Data from one (n=1) participant who has completed the trial showed improvement across several aspects of cognition, as well as sleep is shown in FIGS. 8A-8E. As shown in FIGS. 8A-8E, the disclosed technique combining cognitive training with a music booster showed improvements in each measured category for post-intervention (after using the disclosed technique) versus the participant's baseline. Increases were observed for cognitive flexibility (FIG. 8A) and delayed verbal memory (FIG. 8B) post-intervention. Decreases were observed for sleep onset latency (FIG. 8C), wake time after sleep onset (FIG. 8D), and insomnia severity (FIG. 8E) post-intervention.

Music Listening program improves cognitive function in older adults. A 16-week music listening intervention showed enhanced cognitive performance on measures of inhibition (Stroop task), similar to that exhibited during piano training. Music listening instruction has the capacity to facilitate attention to auditory and visual details in higher order cognitive tasks. This is uniquely provided as a “booster” to cognitive training in the disclosed technique. Pilot experimental data shows improvement in executive function after 6 weeks, further supporting shorter boosters.

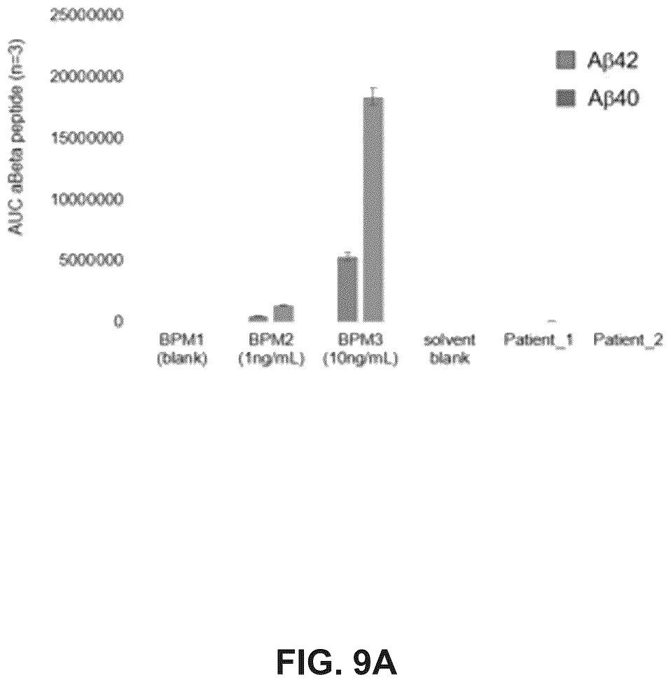

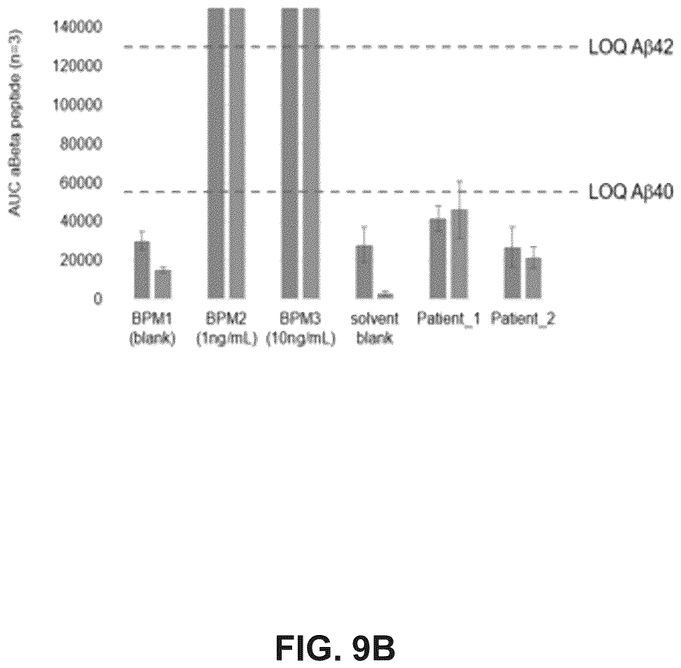

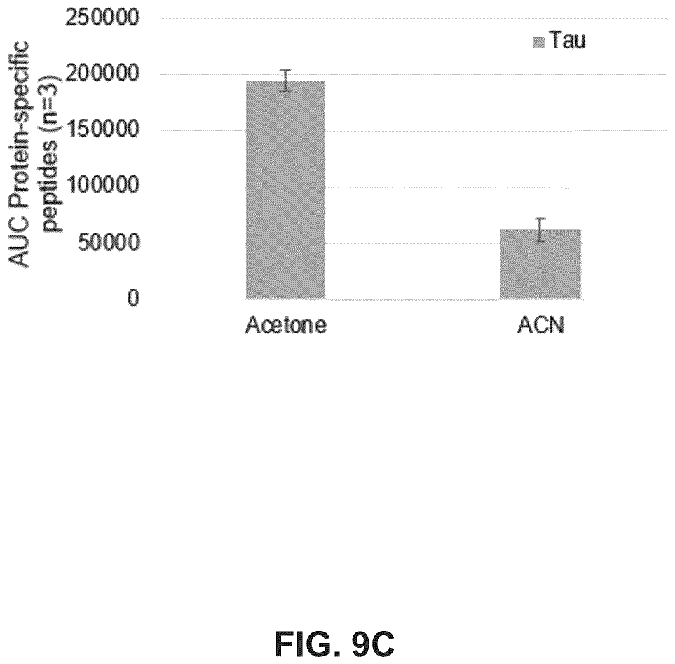

FIGS. 9A-9C are graphs of experimental results for quantification of neurodegeneration biomarkers in plasma.

Feasibility of blood-based inflammatory and neurodegenerative biomarker collection and sample analysis. As shown in FIGS. 9A-9C, quantification of neurodegeneration biomarkers in plasma is feasible in middle-aged and older adults. Participants had chronic widespread pain and/or MCI. Plasma was prepared from whole blood. Plasma protein was extracted using two common techniques: acetone or acetonitrile (ACN) precipitation. One neurodegeneration biomarker (Tau) was quantified after trypsin digestion of extracted protein. Protein-specific peptides were chosen based on sequence (unique to protein) and intensity. Optimized Liquid Chromatography-Multiple Reaction Monitoring (LC-MRM) methods were used to quantify the three proteins in control plasma. Mean and standard deviation (SD) of triplicate injections are shown in FIGS. 9A-9C. Protein recovery for all proteins was significantly better using acetone precipitation. Quantitation of neurodegenerative biomarker Aβ40/42 in plasma has also been shown to be feasible. Plasma was prepared from whole blood and the Aβ40/42 peptides extracted using a standard operating procedure. Three plasma samples were tested: a control (BPM) and two patients. Additionally, two recovery tests were conducted by spiking in commercial standards of Aβ40 and 42 at 1 or 10 ng/mL final concentration in BPM plasma. Aβ40/42 were quantified using LC-MRM on a Thermo Scientific QUANTIVA™ using optimized methods. Signal intensity is shown for Aβ40/42 in samples and spikes and baseline data for the unadulterated control plasma (BPM blank) and the two patients. Limits of quantitation (LOQ) for two peptides are indicated with dashed lines. Mean and SD of triplicate injections are shown.

Example embodiments of the present disclosure implement a multi-phased mixed-method approach to provide a novel combined cognitive training plus music listening intervention (“COGMUSE℠”) to treat older adults with MCI and insomnia. Example embodiments may also be used to treat people with autism, with the music, games, and interface being tailored to the needs of that population and/or to the needs of individual patients. Example embodiments of the present disclosure have potential for high reward as the intervention could be easily disseminated at home, for example, in cognitively vulnerable populations of older adults.

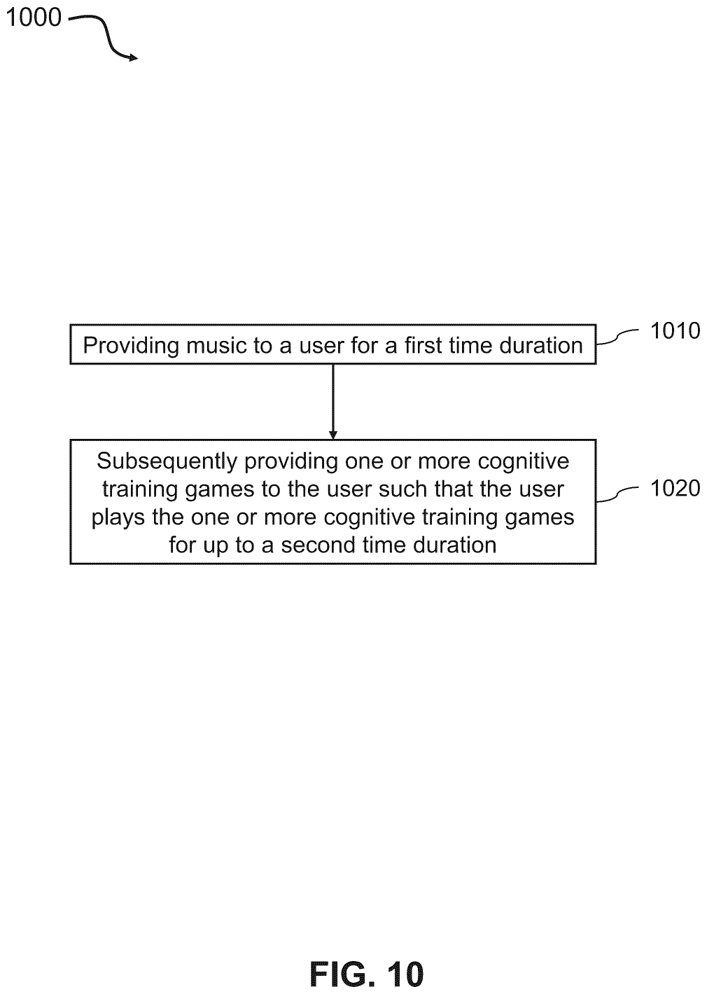

FIG. 10 is a flowchart of an example method for treating cognitive dysfunction.

With reference to FIG. 10, an example method 1000 may include, at 1010, providing music to a user for a first time duration. The example method 1000 may further include, at 1020, subsequently providing one or more cognitive training games to the user such that the user plays the one or more cognitive training games for up to a second time duration.

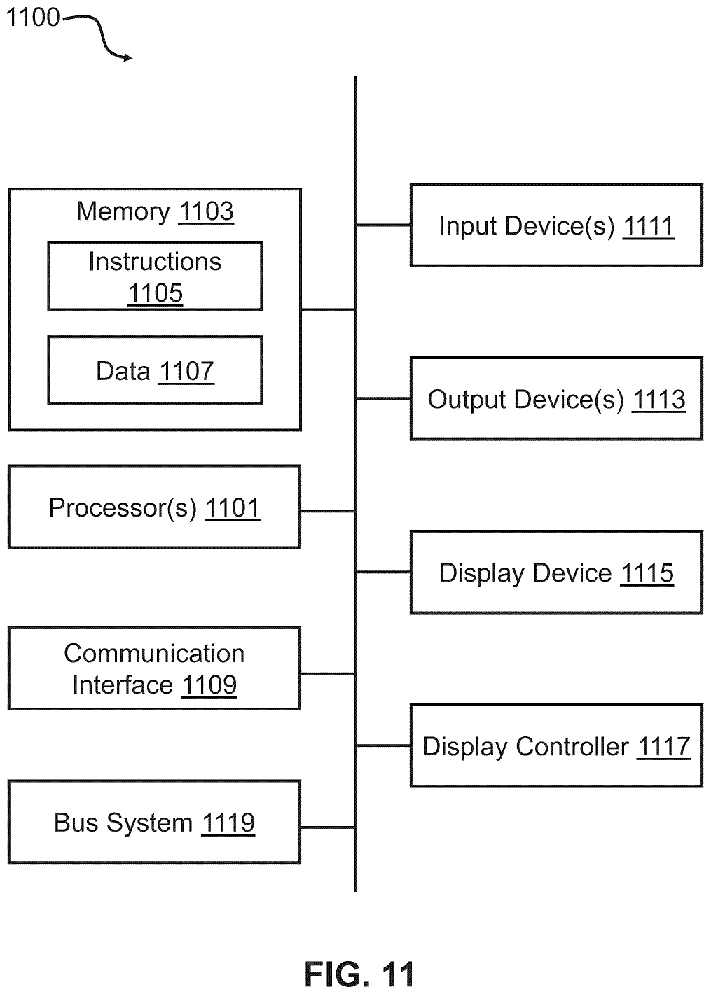

FIG. 11 illustrates certain components that may be included within a computer system according to an example embodiment of the present disclosure.

FIG. 11 illustrates certain components that may be included within a computer system 1100, which may be used to control features according to embodiments of the present disclosure, such as the features discussed with reference to FIGS. 1-10. One or more computer systems 1100 may be used to implement the various devices, components, and systems described herein.

The computer system 1100 includes one or more processors 1101. The processor(s) 1101 may be a single processor or may include multiple processors and/or sub-processors. The processor(s) 1101 may be a general-purpose single- or multi-chip microprocessor (e.g., an Advanced RISC (Reduced Instruction Set Computer) Machine (ARM)), a special-purpose microprocessor (e.g., a digital signal processor (DSP)), a microcontroller, a programmable gate array, etc. The processor(s) 1101 may be referred to as a central processing unit (CPU). Although a single processor(s) 1101 is shown in the computer system 1100 of FIG. 11, in an alternative configuration, a combination of processors (e.g., an ARM and DSP) could be used. In one or more embodiments, the computer system 1100 further includes one or more graphics processing units (GPUs), which can provide processing services related to both entity classification and graph generation.

The computer system 1100 also includes memory 1103 in electronic communication with the processor(s) 1101. The memory 1103 may be any electronic component capable of storing electronic information. For example, the memory 1103 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, at least one non-transitory computer-readable and/or processor-readable medium, and so forth, including combinations thereof. The memory may include a single memory device or multiple memory devices.

Instructions 1105 and data 1107 may be stored in the memory 1103. The instructions 1105 may be executable by the processor(s) 1101 to implement some or all of the functionality disclosed herein. Executing the instructions 1105 may involve the use of the data 1107 that is stored in the memory 1103. Any of the various examples of modules and components described herein may be implemented, partially or wholly, as instructions 1105 stored in memory 1103 and executed by the processor(s) 1101. Any of the various examples of data described herein may be among the data 1107 that is stored in memory 1103 and used during execution of the instructions 1105 by the processor(s) 1101.

A computer system 1100 may also include one or more communication interfaces 1109 for communicating with other electronic devices. The communication interface(s) 1109 may be based on wired communication technology, wireless communication technology, or both. Some examples of communication interfaces 1109 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 1100 may also include one or more input devices 1111 and one or more output devices 1113. Some examples of input devices 1111 include a keyboard, mouse, microphone, remote control device, button, joystick, trackball, touchpad, and lightpen. Some examples of output devices 1113 include a speaker and a printer. One specific type of output device that is typically included in a computer system 1100 is a display device 1115. Display devices 1115 used with embodiments disclosed herein may utilize any suitable image projection technology, such as liquid crystal display (LCD), light-emitting diode (LED), gas plasma, electroluminescence, or the like. A display controller 1117 may also be provided, for converting data 1107 stored in the memory 1103 into text, graphics, and/or moving images (as appropriate) shown on the display device 1115.

The various components of the computer system 1100 may be coupled together by one or more buses, which may include a power bus, a control signal bus, a status signal bus, a data bus, etc. For the sake of clarity, the various buses are illustrated in FIG. 11 as a bus system 1119.

The following are sections in accordance with at least one embodiment of the present disclosure:

Clause 1: A method for treating cognitive dysfunction, the method including: providing music to a user for a first time duration, and subsequently providing one or more cognitive training games to the user such that the user plays each of the one or more cognitive training games for up to a second time duration.

Clause 2: The method of clause 1, wherein the method is performed at least two hours before the user goes to bed.

Clause 3: The method of clause 1, wherein the first time duration is 15 minutes.

Clause 4: The method of clause 1, wherein the second time duration is 15 minutes or 20 minutes.

Clause 5: The method of clause 1, wherein the one or more cognitive training games are configured to provide training for multi-domain attention, processing speed, and executive function in the user.

Clause 6: The method of clause 1, wherein the one or more cognitive training games are configured to provide training to the user for one or more of: processing speed, attention, perceptual-motor skill, inhibition, visuospatial working memory, spatial attention, or perceptual-motor skills.

Clause 7: The method of clause 6, wherein the one or more cognitive training games are configured to provide training to the user corresponding to groups of skills, the groups including one or more of: processing speed, attention, perceptual-motor skill, and inhibition, processing speed, attention, visuospatial working memory, and inhibition, processing speed, attention, visuospatial working memory, and inhibition, or spatial attention, perceptual-motor skills, processing speed, and inhibition.

Clause 8: The method of clause 1, wherein the one or more cognitive training games are provided as a plurality of cognitive training games, each of the plurality of cognitive training games being configured to train a user in a different grouping of one or more skills.

Clause 9: The method of clause 1, further including: receiving a user training result quantifying cognitive dysfunction treatment result from performing the method, and based on the received user training result, automatically changing one or more of a type of the music, the first time duration, the one or more cognitive training games, or the second time duration.

Clause 10: The method of clause 9, wherein the user training result and the automatic changing are performed iteratively until a threshold treatment result has been achieved.

Clause 11: A system for treating cognitive dysfunction, including: one or more processors, and at least one memory including at least one non-transitory computer-readable medium storing instructions that, when executed by at least one of the one or more processors, cause the system to perform operations, the operations including: providing music to a user for a first time duration, and subsequently providing one or more cognitive training games to the user such that the user plays the one or more cognitive training games for up to a second time duration.

Clause 12: The system of clause 11, wherein the operations are performed at least two hours before the user goes to bed.

Clause 13: The system of clause 11, wherein the first time duration is 15 minutes.

Clause 14: The system of clause 11, wherein the second time duration is 15 minutes or 20 minutes.

Clause 15: The system of clause 11, wherein the one or more cognitive training games are configured to provide training for multi-domain attention, processing speed, and executive function in the user.

Clause 16: The system of clause 11, wherein the one or more cognitive training games are configured to provide training to the user for one or more of: processing speed, attention, perceptual-motor skill, inhibition, visuospatial working memory, spatial attention, or perceptual-motor skills.

Clause 17: The system of clause 16, wherein the one or more cognitive training games are configured to provide training to the user corresponding to groups of skills, the groups including one or more of: processing speed, attention, perceptual-motor skill, and inhibition, processing speed, attention, visuospatial working memory, and inhibition, processing speed, attention, visuospatial working memory, and inhibition, or spatial attention, perceptual-motor skills, processing speed, and inhibition.

Clause 18: The system of clause 11, wherein the one or more cognitive training games are provided as a plurality of cognitive training games, each of the plurality of cognitive training games being configured to train a user in a different grouping of one or more skills.

Clause 19: The system of clause 11, further including: receiving a user training result quantifying cognitive dysfunction treatment result from performing the operations, and based on the received user training result, automatically changing one or more of a type of the music, the first time duration, the one or more cognitive training games, or the second time duration.

Clause 20: A non-transitory computer-readable medium for treating cognitive dysfunction, the non-transitory computer-readable medium storing instructions that, when executed by at least one processor of a computing system, cause the computing system to perform operations, the operations including: providing music to a user for a first time duration, and subsequently providing one or more cognitive training games to the user such that the user plays the one or more cognitive training games for up to a second time duration.

Systems and software, e.g., implemented on a non-transitory computer-readable medium, for performing the methods discussed herein are also within the scope of embodiments of the present disclosure.

Embodiments of the present disclosure may thus utilize a special purpose or general-purpose computing system including computer hardware, such as, for example, one or more processors and system memory. Embodiments within the scope of the present disclosure also include physical and other computer-readable media for carrying or storing computer-executable instructions and/or data structures, including applications, tables, data, libraries, or other modules used to execute particular functions or direct selection or execution of other modules. Such computer-readable media can be any available media that can be accessed by a general purpose or special purpose computer system. Computer-readable media that store computer-executable instructions (or software instructions) are physical storage media. Computer-readable media that carry computer-executable instructions are transmission media. Thus, by way of example, and not limitation, embodiments of the present disclosure can include at least two distinctly different kinds of computer-readable media, namely physical storage media or transmission media. Combinations of physical storage media and transmission media should also be included within the scope of computer-readable media.

Both physical storage media and transmission media may be used temporarily store or carry software instructions in the form of computer readable program code that allows performance of embodiments of the present disclosure. Physical storage media may further be used to persistently or permanently store such software instructions. Examples of physical storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored as or in software, hardware, firmware, or combinations thereof.

A “network” or “communications network” may generally be defined as one or more data links that enable the transport of electronic data between computer systems and/or modules, engines, and/or other electronic devices. When information is transferred or provided over a communication network or another communications connection (either hardwired, wireless, or a combination of hardwired or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instructions or data structures and which can be accessed by a general purpose or special purpose computer.

Further, upon reaching various computer system components, program code in the form of computer-executable instructions or data structures can be transferred automatically or manually from transmission media to physical storage media (or vice versa). For example, computer-executable instructions or data structures received over a network or data link can be buffered in memory (e.g., RAM) within a network interface module (NIC), and then eventually transferred to computer system RAM and/or to less volatile physical storage media at a computer system. Thus, it should be understood that physical storage media can be included in computer system components that also (or even primarily) utilize transmission media.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

As used in this specification and the claims, the singular forms “a,” “an,” and “the” include plural forms unless the context clearly dictates otherwise. The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims. Any trademarks or service marks mentioned herein are the property of their respective owners.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter. Any trademarks are the property of their respective owners.

The phrase “such as” should be interpreted as “for example, including.” Moreover, the use of any and all example language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for treating cognitive dysfunction, the method comprising:
providing music to a user for a first time duration; and
subsequently providing one or more cognitive training games to the user such that the user plays each of the one or more cognitive training games for up to a second time duration.

2. The method of claim 1, wherein the method is performed at least two hours before the user goes to bed.

3. The method of claim 1, wherein the first time duration is 15 minutes.

4. The method of claim 1, wherein the second time duration is 15 minutes or 20 minutes.

5. The method of claim 1, wherein the one or more cognitive training games are configured to provide training for multi-domain attention, processing speed, and executive function in the user.

6. The method of claim 1, wherein the one or more cognitive training games are configured to provide training to the user for one or more of: processing speed, attention, perceptual-motor skill, inhibition, visuospatial working memory, spatial attention, or perceptual-motor skills.

7. The method of claim 6, wherein the one or more cognitive training games are configured to provide training to the user corresponding to groups of skills, the groups comprising one or more of:
processing speed, attention, perceptual-motor skill, and inhibition;
processing speed, attention, visuospatial working memory, and inhibition;
processing speed, attention, visuospatial working memory, and inhibition; or
spatial attention, perceptual-motor skills, processing speed, and inhibition.

8. The method of claim 1, wherein the one or more cognitive training games are provided as a plurality of cognitive training games, each of the plurality of cognitive training games being configured to train a user in a different grouping of one or more skills.

9. The method of claim 1, further comprising:
receiving a user training result quantifying cognitive dysfunction treatment result from performing the method; and
based on the received user training result, automatically changing one or more of a type of the music, the first time duration, the one or more cognitive training games, or the second time duration.

10. The method of claim 9, wherein the user training result and the automatic changing are performed iteratively until a threshold treatment result has been achieved.

11. A system for treating cognitive dysfunction, comprising:
one or more processors; and
at least one memory comprising at least one non-transitory computer-readable medium storing instructions that, when executed by at least one of the one or more processors, cause the system to perform operations, the operations comprising:
providing music to a user for a first time duration; and
subsequently providing one or more cognitive training games to the user such that the user plays the one or more cognitive training games for up to a second time duration.

12. The system of claim 11, wherein the operations are performed at least two hours before the user goes to bed.

13. The system of claim 11, wherein the first time duration is 15 minutes.

14. The system of claim 11, wherein the second time duration is 15 minutes or 20 minutes.

15. The system of claim 11, wherein the one or more cognitive training games are configured to provide training for multi-domain attention, processing speed, and executive function in the user.

16. The system of claim 11, wherein the one or more cognitive training games are configured to provide training to the user for one or more of: processing speed, attention, perceptual-motor skill, inhibition, visuospatial working memory, spatial attention, or perceptual-motor skills.

17. The system of claim 16, wherein the one or more cognitive training games are configured to provide training to the user corresponding to groups of skills, the groups comprising one or more of:
processing speed, attention, perceptual-motor skill, and inhibition;
processing speed, attention, visuospatial working memory, and inhibition;
processing speed, attention, visuospatial working memory, and inhibition; or
spatial attention, perceptual-motor skills, processing speed, and inhibition.

18. The system of claim 11, wherein the one or more cognitive training games are provided as a plurality of cognitive training games, each of the plurality of cognitive training games being configured to train a user in a different grouping of one or more skills.

19. The system of claim 11, further comprising:
receiving a user training result quantifying cognitive dysfunction treatment result from performing the operations; and
based on the received user training result, automatically changing one or more of a type of the music, the first time duration, the one or more cognitive training games, or the second time duration.

20. A non-transitory computer-readable medium for treating cognitive dysfunction, the non-transitory computer-readable medium storing instructions that, when executed by at least one processor of a computing system, cause the computing system to perform operations, the operations comprising:
providing music to a user for a first time duration; and
subsequently providing one or more cognitive training games to the user such that the user plays the one or more cognitive training games for up to a second time duration.
